(19) United States
(12) Patent Application Publication
(10) Pub. No.: US 2021/0059720 A1
(43) Pub. Date: Mar. 4, 2021

(54) CRANIAL DECOMPRESSION CONNECTOR

(71) Applicant: Guo JIANG, Gulou Nanjing, Jiangsu (CN)

(72) Inventor: Guo JIANG, Gulou Nanjing, Jiangsu (CN)

(21) Appl. No.: 16/982,466

(22) PCT Filed: Oct. 19, 2018
(86) PCT No.: PCT/CN2018/110974

§ 371 Date: Sep. 18, 2020

(30) Foreign Application Priority Data
Mar. 20, 2018 (CN) 201820377606.0

(51) Int. Cl. A61B 17/68; A61B 17/80; A61B 17/84

(52) U.S. Cl. CPC A61B 17/688; A61B 17/842; A61B 17/8061

(57) ABSTRACT

Disclosed is a cranial decompression connector, including two screws (1) and an elastic connecting member (2) connected between the two screws (1). Two ends of the elastic connecting member (2) are respectively connected to the two screws (1), one screw (1) is configured to be fixedly connected to a bone window, and the other screw (1) is configured to be fixedly connected to a bone plate. When a patient undergoes a craniotomy, the two screws (1) are respectively fixed to the bone window and the bone plate, and the two ends of the elastic connecting member (2) are respectively connected to the two screws (1) to connect the bone window and the bone plate together. As the pressure of the patient's brain tissue increases, the bone plate is displaced to the outside and protrudes out of the bone window to decrease the intracranial pressure, the patient's brain tissue swelling subsides, the intracranial pressure returns to the normal range, and the bone plate automatically returns to the original position under the traction of the elastic connecting member (2), such that the bone plate gradually merges with the bone window to form a whole. The self-repair of the skull is achieved, and secondary skull repair for the patient is avoided.

### BACKGROUND

### Technical Field

The present invention relates to a cranial decompression connector which is suitable for use by patients undergoing craniocerebral decompression, and belongs to the technical field of medical instruments.

### Related Art

Most patients suffering cerebral traumas, cerebral hemorrhage and various intracranial lesions require a craniotomy. Patients often experience brain tissue swelling after cerebral hemorrhage and tumor surgeries. At this time, a skull plate needs to be removed, and the cut skull plate is usually discarded, which artificially causes local skull defect, usually called a bone window. With the bone window of the skull defect, the swollen brain tissue can protrude out of the bone window when the pressure increases to decrease the intracranial pressure and reduce the degree of pressure on important tissues in the brain. In particular, patients suffering cerebral traumas often have severe brain tissue swelling, so it is necessary to remove the skull plate for decompression, so as to prevent the patients from experiencing cerebral hernia and causing death. The more severe the patient's brain tissue swelling, the larger the skull plate that needs to be removed. This is an extremely important method for rescuing patients suffering brain swelling. Patients suffering head traumas have brain tissue swelling, and the increase in intracranial pressure is a transient pathological process. In general, after two to three weeks after the surgery, that is, in the later stage of cerebral traumas, the patient's brain tissue swelling disappears, and the brain tissue protruding out of the skull window retracts into the skull cavity. As the intracranial pressure becomes lower and lower and the standing and lying positions change, the imbalance of intracranial and extracranial pressure is caused. The brain tissue of the skull defect part is subject to change in body position pressure to protrude out of the hone window and recess into the bone window from time to time, which causes herniation of the brain tissue, thereby easily causing softening and necrosis of the brain tissue of this part below the bone window. Therefore, artificial tissues are needed to repair the skull to protect brain tissues. This not only increases the patient's pain during the surgery, but also increases the patient's economic burden. The prior art lacks a medical instrument which can enable patients to achieve the effect of intracranial decompression and avoid secondary skull repair, which is a great boon for patients who need to undergo craniocerebral decompression.

### SUMMARY

The objective of the present invention is to provide a cranial decompression connector which can enable patients undergoing a craniotomy to achieve the effect of intracranial decompression and avoid secondary skull repair.

The present invention adopts the following technical solution: a cranial decompression connector includes two screws and an elastic connecting member connected between the two screws. Two ends of the elastic connecting member are respectively connected to the two screws, one screw is configured to be fixedly connected to a bone window, and the other screw is configured to be fixedly connected to a bone plate.

The screw includes an external screw with a hollow inside and an internal screw with external threads on the outside. A hollow part of the external screw is provided with internal threads. The internal screw is in threaded connection with the hollow part of the external screw. The end of the elastic connecting member passes through the hollow part of the external screw and is clamped between the internal screw and the external screw.

The external screw is a cancellous screw.

The cranial decompression connector further includes two connecting sheets. Each of the connecting sheets is provided with a screw through hole and a connecting member through hole. The screw is inserted into the screw through hole. The elastic connecting member is fixed to the connecting member through hole.

The two ends of the elastic connecting member are respectively wound on the two screws.

The two ends of the elastic connecting member are respectively provided with a fixing ring, and the fixing ring is sleeved over the screw.

The screw is a cancellous screw.

The elastic connecting member is an elastic silicone band or an elastic rubber band.

The elastic connecting member is a spring.

The spring is made of a stainless steel wire or a titanium alloy wire.

The present invention has the following beneficial effects: when a patient undergoes a craniotomy, the cut skull bone plate does not need to be discarded, the two screws are respectively fixed to the bone window and the bone plate, and the two ends of the elastic connecting member are respectively connected to the two screws to connect the bone window and the bone plate together. Because the bone window and the bone plate are connected by the elastic connecting member, as the patient's brain tissue swells and the pressure increases, the bone plate at the bone window is displaced to the outside and protrudes out of the bone window, thereby increasing the volume of the skull window brain tissue, decreasing the intracranial pressure, and preventing the formation of cerebral hernia. As time goes by and the condition improves, the patient's brain tissue swelling subsides, the intracranial pressure returns to the normal range, and the brain tissue no longer protrudes out of the bone window. Under the traction of the elastic connecting member, the movable skull bone plate automatically returns to the original position, and the bone window is fixed. After a long time, the bone plate grows in situ and gradually merges with the bone window to form a whole. Thus, the self-repair of the skull plate is achieved, and secondary skull repair for the patient is avoided.

Preferably, the elastic connecting member is clamped by the external screw and the internal screw, which has the characteristics of simple structure and convenient operation.

Preferably, during use, after the screws are installed in the connecting member through hole, the screws are fixed to the bone window or the bone plate, the end of the elastic connecting member is fixed in the connecting member through hole, and the operation is more convenient through the connecting sheets.

Preferably, during use, the fixing rings may be sleeved over the screws, and then, the screws are fixed to the bone window or the bone plate.

Preferably, during use, the end of the elastic connecting member may be wound outside the screws.

Preferably, cancellous screws are adopted to adapt to the skull surgery.

Preferably, the elastic silicone band or rubber band is adopted to enable the patient to feel more comfortable and cause less harm to the human body.

Preferably, the elastic connecting member is the spring having the characteristics of large elasticity and good decompression effect.

### BRIEF DESCRIPTION OF THE DRAWINGS

FIG. 1 is a schematic structural diagram of an embodiment 1 of the present invention;

FIG. 2 is a use state diagram I of the embodiment 1;

FIG. 3 is a use state diagram II of the embodiment 1;

FIG. 4 is a use state diagram III of the embodiment 1;

FIG. 5 is a use state diagram of an embodiment 2 of the present invention;

FIG. 6 is a schematic structural diagram of a connecting sheet in the embodiment 2; and

FIG. 7 is a schematic structural diagram of an embodiment 3 of the present invention.

In the drawings, 1 denotes a screw, 2 denotes an elastic connecting member, 3 denotes a connecting sheet, 4 denotes a skull bone plate, 5 denotes a concentric bone ring, 6 denotes a bone window, 11 denotes an external screw, 12 denotes an internal screw, 31 denotes a screw through hole, and 32 denotes a connecting member through hole.

### DETAILED DESCRIPTION

The present invention will be described in detail below in conjunction with the accompanying drawings and specific embodiments.

### Embodiment 1

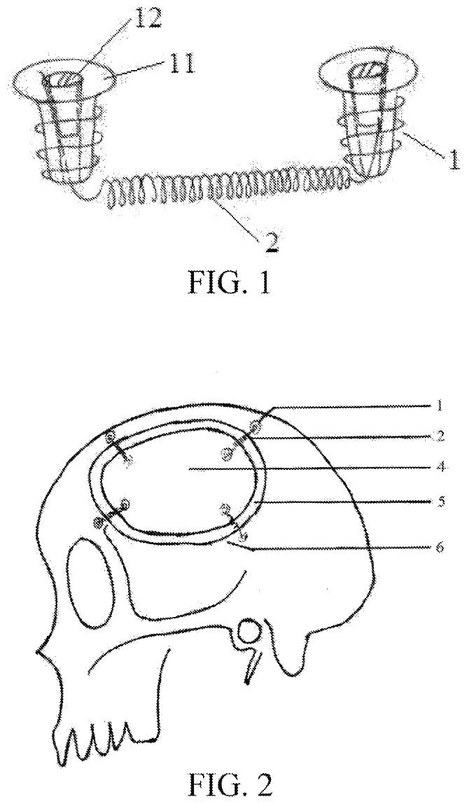

The structure of an embodiment 1 of the present invention is shown in FIG. 1 to FIG. 4.

The cranial decompression connector of the present embodiment includes two screws 1 and an elastic connecting member 2 connected between the two screws 1. The elastic connecting member 2 of the present embodiment is a spring, and the spring is made of a stainless steel wire or a titanium alloy wire. Two ends of the elastic connecting member 2 are respectively connected to the two screws 1, one screw is configured to be fixedly connected to a bone window, and the other screw is configured to be fixedly connected to a bone plate. The screw 1 includes an external screw 11 with a hollow inside and an internal screw 12 with external threads on the outside. A hollow part of the external screw 11 is provided with internal threads. The internal screw 12 is in threaded connection with the hollow part of the external screw 11. The end of the elastic connecting member 2 passes through the hollow part of the external screw and is clamped between the internal screw 12 and the external screw 11. The external screw 11 is a cancellous screw.

To achieve the above, it may be necessary to meet three conditions: firstly, the bone plate cut from a decompression window can easily protrude to the outside after being put in; secondly, the bone plate of the decompression window and the edge of the decompression window necessarily have a good dynamic connection; and thirdly, the bone plate at the decompression window returns to the normal position, but cannot recess into the skull.

When a patient needs the craniotomy because of craniocerebral traumas or intracranial hematomas, it is normal to drill a hole with a wire saw or mill out a bone block in a suitable size with a milling cutter, and a bone window formed by removing the bone block is the decompression window. At the end of the craniotomy, the bone plate is reset, and a certain gap is reserved, so that the hone plate protrudes out of the window so as to achieve an effect of increasing the volume of the cranial cavity. However, due to the limitation of the scalp tissue, it is impossible for one bone joint to have too large gap, which cannot meet the requirement of decompression, Another ring cutting may be made inside the outer edge of the bone plate to cut a concentric bone ring. A part of the bone plate protrudes to the outside to increase the degree of protrusion and increase the volume of the cranial cavity. If necessary, a second cutting line may be made to form concentric ring cutting to meet the requirement of increasing the intracranial volume. The concentric ring cutting method can adapt to the requirement of increasing the intracranial volume. For the dynamic connection between the edge of the decompression window and the skull plate and the concentric bone ring, a tiny stainless steel spring is selected as the elastic connecting member. The skull is composed of internal and external plates and a diploe between the internal and external plates. The internal and external plates of the skull are harder. The diploe between the internal and external plates is looser and the thickness of the diploe is in a range of 6 to 8 mm. A transverse hole of about 3 mm may be made in the diploe between the internal and external plates, and the spring is put in the transverse hole to serve as a connecting component among the skull bone plate, the concentric bone ring and the skull bone window. Three to five connections may be placed on the periphery of each bone plate, and the two ends of the spring are fixed with special fixing screws. The skull plate reset in this way can be displaced and reset outward with the increase and decrease of the intracranial pressure, thereby effectively achieving the effect of decreasing the intracranial pressure. In order to prevent the skull plate from entering the cranial cavity, two measures are mainly taken. Firstly, when the expansion joint of the skull plate is cut, methods and tools that make the joint of the skull plate smaller are used as much as possible, and there are three common methods: (1) an ordinary wire saw is used, and a wire saw with a smaller diameter is used as much as possible; (2) a laser method is used to cut the skull plate, and the joint can be adjusted; and (3) an ultrasonic bone cutting method is used for cutting. Secondly, during bone cutting, the bone is inclined outward at a certain angle to form a wedge with a small inside and large outside, the skull bone plate cannot enter the skull, and a normal outward-inclined angle a is greater than or equal to 5 degrees.

Specific operation processes are as follows: when a patient needs to undergo decompression, a laser cutting machine is used to cut a 3 to 6 mm wide ring on the cut bone plate from the edge of the original bone plate, so as to generate a second decompression joint for the free bone plate. The incision is slightly inclined to the outside at a certain angle, so that the bone plate in the middle of the concentric bone ring can only move to the outside, but not to the inside. In addition, the joint of the incision should be as thin as possible to enable a made slope internal stop which can effectively prevent the skull plate at the middle part from inward recessing into the cranial cavity, and also reduce the gap of the skull defect to make the skull grow smoothly. The bone cutting method may further be performed by using an ultrasonic bone cutter or a wire saw or a milling cutter. When the concentric bone ring of the skull plate is cut, only one concentric bone ring or a plurality of concentric bone rings may be cut. The more concentric bone rings cut, the greater the degree of swelling of the bone plate to the outside. Similarly, each incision has a certain outward-inclined angle to prevent the bone plate in the inside part from slipping into the cranial cavity.

Connection of skull plate, bone ring and skull: the cut skull plate is put in the part of the skull bone window, three or four thicker parts of the skull are selected as connecting points between the free bone plate and the bone window, and are marked, then, a hole is drilled at the part of the diploe between the internal and external plates, a tiny spring is put in this hole, and the spring serves as the connecting component among the bone plate, the concentric bone ring and the bone window. The internal and external ends of the spring are respectively fixed by the external screws, and the external screws have cancellous threads on the outside and mechanical threaded holes on the inside. During specific operation, firstly, a small hole is drilled on the edge of the bone window and is communicated with the hole drilled on the diploe, the spring wire is led out from the mechanical threaded hole of the external screw, the fixing screw is fixed to the skull, and then, a smaller internal bolt is screwed into the mechanical threaded hole to fix the spring wire and cut off the excess spring wires. Therefore, a point is fixed successfully. The same method may be used to fix other points.

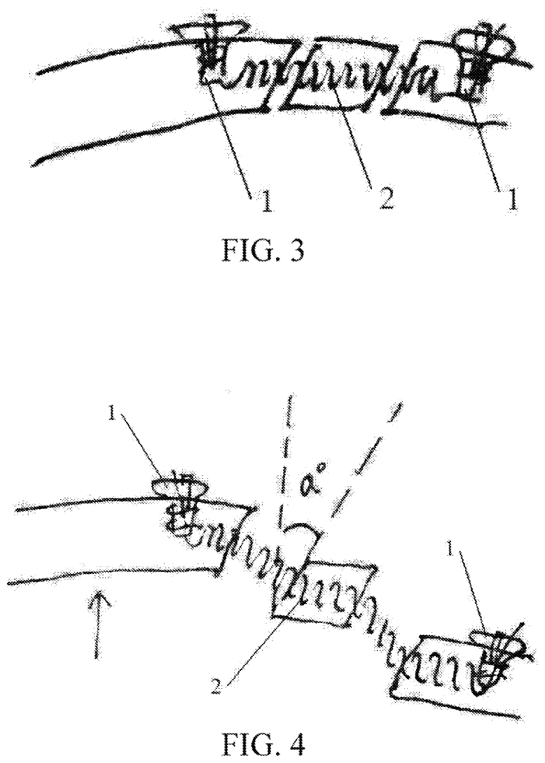

The screw in the present embodiment can realize self-locking fixation. As shown in one end of the external screw is provided with a big-end cross groove, the outside of other end is provided with cancellous threads, the middle is provided with a hole with mechanical threads, and after the spring wire is threaded out from the inside to the outside, the middle is tightened in the threaded hole in the external screw by using the internal screw so as to fix the spring. FIG. 3 is a schematic diagram of connection of the free bone plate 4, the concentric bone ring 5 and the edge of the skull bone window 6 by a spring, and is also a state diagram of the bone plate resetting. FIG. 4 is a schematic diagram of connection of the free bone plate 4, the concentric bone ring 5 and the edge of the skull bone window 6 by a spring and is also a state diagram of the bone plate protruding to the outside when the brain tissue swells, and also shows that the angle a of the external bevel cut from the bone ring is greater than or equal to 5 degrees.

During use of the cranial decompression connector of the present invention, because the bone window and the bone plate are connected by the elastic connecting member, as the patient's brain tissue swells and the pressure increases, the bone plate at the bone window is displaced to the outside and protrudes out of the bone window, thereby increasing the volume of the skull window brain tissue, decreasing the intracranial pressure, and preventing the formation of cerebral hernia. As time goes by and the condition improves, the patient's brain tissue swelling subsides, the intracranial pressure returns to the normal range, and the brain tissue no longer protrudes out of the bone window. Under the traction of the elastic connecting member, the movable skull bone plate automatically returns to the original position, and the bone window is fixed. After a long time, the bone plate grows in situ and gradually merges with the bone window to form a whole. Thus, the self-repair of the skull plate is achieved, and secondary skull repair for the patient is avoided.

### Embodiment 2

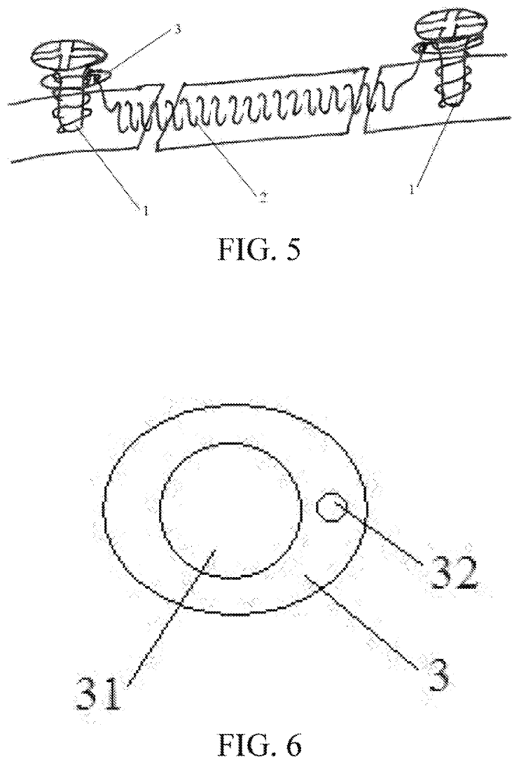

The difference from the embodiment 1 of the present invention is that the connection mode of screws and an elastic connecting member in the present embodiment is different. In the present embodiment, connecting sheets are adopted to connect the screws with the elastic connecting member. The structure of the embodiment 2 of the present invention is shown in FIG. 5 to FIG. 6. A cranial decompression connector of the present embodiment further includes two connecting sheets 3. Each of the connecting sheets 3 is provided with a screw through hole 31 and a connecting member through hole 32. The screw 1 is inserted into the screw through hole 31. The elastic connecting member 2 is fixed to the connecting member through hole 32. The screw 1 is a cancellous screw. The other parts in the present embodiment are the same as those in the embodiment 1, and are not repeated herein.

When a patient undergoes a craniotomy, the cut skull bone plate does not need to be discarded, the two screws respectively pass through the screw through holes on the two connecting sheets, then the two screws are respectively fixed to the bone window and the bone plate, and the two ends of the spring are respectively fixed to the connecting member through holes on the two connecting sheets to connect the bone window and the bone plate together.

### Embodiment 3

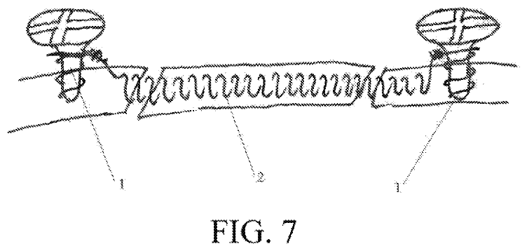

The difference from the embodiment 1 of the present invention is that the connection mode of screws and an elastic connecting member in the present embodiment is different. The structure of the embodiment 3 of the present invention is shown in FIG. 7. In the present embodiment, two ends of the elastic connecting member 2, that is, a spring, are respectively wound on the two screws 1, and the screw 1 is a cancellous screw. The other parts in the present embodiment are the same as those in the embodiment 1, and are not repeated herein.

When a patient undergoes a craniotomy, the cut skull bone plate does not need to be discarded, the two ends of the elastic connecting member are respectively wound on the two screws, and then, two external screws are respectively fixed to the bone window and the bone plate to connect the bone window and the bone plate together.

In other embodiments of the present invention, the two ends of the elastic connecting member 2 may be respectively provided with a fixing ring. When a patient undergoes a craniotomy, the fixing rings at the two ends of the elastic connecting member are respectively sleeved over two screws, and then, two external screws are respectively fixed to the bone window and the bone plate to connect the bone window and the bone plate together.

The above embodiments are exemplary embodiments of the present invention. In other embodiments of the present invention, the elastic connecting member may further be an elastic silicone band or an elastic rubber band.

1. A cranial decompression connector, comprising two screws (1) and an elastic connecting member (2) connected between the two screws (1), wherein two ends of the elastic connecting member (2) are respectively connected to the two screws (1), one screw is configured to be fixedly connected to a bone window, the other screw is configured to be fixedly connected to a bone plate, and the elastic connecting member is configured to be disposed in a transverse hole within a skull diploe to serve as a connecting component among the skull bone plate, a concentric bone ring and the skull bone window.

2. The cranial decompression connector according to claim 1, wherein the screw (1) comprises an external screw (11) with a hollow inside and an internal screw (12) with external threads on the outside, a hollow part of the external screw (11) is provided with internal threads, the internal screw (12) is in threaded connection with the hollow part of the external screw (11), and the end of the elastic connecting member (2) passes through the hollow part of the external screw and is clamped between the internal screw (12) and the external screw (11).

3. The cranial decompression connector according to claim 2, wherein the external screw (11) is a cancellous screw.

4. The cranial decompression connector according to claim 1, further comprising two connecting sheets (3), each of the connecting sheets (3) is provided with a screw through hole (31) and a connecting member through hole (32), the screw (1) is inserted into the screw through hole (31), and the elastic connecting member (2) is fixed to the connecting member through hole (32).

5. The cranial decompression connector according to claim 1, wherein the two ends of the elastic connecting member (2) are respectively wound on the two screws (1).

6. The cranial decompression connector according to claim 1, wherein the two ends of the elastic connecting member (2) are respectively provided with a fixing ring, and the fixing ring is sleeved over the screw (1).

7. The cranial decompression connector according to claim 4, wherein the screw (1) is a cancellous screw.

8. The cranial decompression connector according to claim 1, wherein the elastic connecting member (2) is an elastic silicone band, an elastic rubber band, or a spring.

9. (canceled)

10. The cranial decompression connector according to claim 9, wherein the spring is made of a stainless steel wire or a titanium alloy wire.

11. The cranial decompression connector according to claim 5, wherein the screw (1) is a cancellous screw.

12. The cranial decompression connector according to claim 6, wherein the screw (1) is a cancellous screw.

13. The cranial decompression connector according to claim 2, wherein the elastic connecting member (2) is an elastic silicone band, an elastic rubber band, or a spring.

14. The cranial decompression connector according to claim 3, wherein the elastic connecting member (2) is an elastic silicone band, an elastic rubber band, or a spring.

15. The cranial decompression connector according to claim 4, wherein the elastic connecting member (2) is an elastic silicone band, an elastic rubber band, or a spring.

16. The cranial decompression connector according to claim 5, wherein the elastic connecting member (2) is an elastic silicone band, an elastic rubber band, or a spring.

17. The cranial decompression connector according to claim 6, wherein the elastic connecting member (2) is an elastic silicone band, an elastic rubber band, or a spring.
